(19) United States
(12) Patent Application Publication
(10) Pub. No.: US 2023/0081412 A1
(43) Pub. Date: Mar. 16, 2023

(54) APPLYING A LAYERED APPROACH TO DETERMINING MOLECULAR RETROSYNTHETIC ROUTE USING A NEURAL NETWORK

(71) Applicant: Tencent Technology (Shenzhen) Company Limited, Shenzhen (CN)

(72) Inventors: Yue FU, Shenzhen (CN); Chang-Yu HSIEH, Shenzhen (CN); Benben LIAO, Shenzhen (CN); Jianye HAO, Shenzhen (CN); Shengyu ZHANG, Shenzhen (CN)

(73) Assignee: Tencent Technology (Shenzhen) Company Limited, Shenzhen (CN)

(21) Appl. No.: 17/986,559

(22) Filed: Nov. 14, 2022

Related U.S. Application Data
(63) Continuation of application No. PCT/CN2021/122724, filed on Oct. 9, 2021.

(30) Foreign Application Priority Data
Nov. 4, 2020 (CN) 202011218991.2

(51) Int. Cl. G16C 20/70; G06N 3/04; G06N 3/08; G16C 20/10

(52) U.S. Cl. CPC G16C 20/70; G06N 3/0454; G06N 3/08; G16C 20/10

(57) ABSTRACT

A training method for a neural network includes determining first disassembly paths of a plurality of first molecules, and obtaining a first cost dictionary based on the first disassembly paths of the first molecules. The method also includes determining molecular expression information of second molecules based on the first disassembly paths of the first molecules, and determining a plurality of third molecules from the second molecules, each of the third molecules representing a class of the second molecules. The method further includes obtaining a second cost dictionary based on second disassembly paths of the third molecules, and performing training based on the first cost dictionary and the second cost dictionary to obtain a target neural network. The target neural network being configured to output cost value information corresponding to a target molecule according to input molecular expression information of the target molecule.

### RELATED APPLICATIONS

This application is a continuation of International Application No. PCT/CN2021/122724, entitled “METHOD AND APPARATUS FOR TRAINING NEURAL NETWORK FOR DETERMINING MOLECULE RETROSYNTHESIS ROUTE” and filed on Oct. 9, 2021, which claims priority to Chinese Patent Application No. 202011218991.2, entitled “TRAINING METHOD AND APPARATUS FOR NEUTRAL NETWORK FOR DETERMINING MOLECULAR RETROSYNTHETIC ROUTE” filed on Nov. 4, 2020. The entire disclosures of the prior applications are hereby incorporated by reference.

### FIELD OF THE TECHNOLOGY

This application relates to the technical field of artificial intelligence, including a training method and apparatus for a neural network for determining a molecular retrosynthetic route, and equipment and a readable storage medium.

### BACKGROUND OF THE DISCLOSURE

In recent years, with the rapid development of artificial intelligence technology, it has been gradually introduced into and plays an important role in various scientific areas. In the field of chemistry, since chemical reactions are endlessly variable under different conditions, a reasonable organic synthetic route is needed to be designed by researchers, which consumes a lot of time and efforts when compound molecules are prepared in the past. Where organic synthetic routes are designed by researchers with the aid of artificial intelligence technology, efficiency in developing chemical drug molecules and other compounds by researchers can be greatly improved.

The current methods for designing molecular retrosynthetic routes based on artificial intelligence include the following. One method includes a random search step based on the Monte Carlo Tree Search (MCTS) algorithm until a solution is found or a maximum depth is reached, and symbolic artificial intelligence is introduced to complete the design of the molecular retrosynthetic route. Another method is to determine a template selection strategy for each step of the molecular retrosynthetic reaction based on deep reinforcement learning technology, and finally obtain a molecular retrosynthetic route. Another method is to use a distributed training architecture in combination with deep reinforcement learning technology to accelerate the construction of an optimal molecular retrosynthetic route and the fitting of a network of a cost value function, and implement the design of a training set molecular retrosynthetic route through the network.

However, it takes a long time to design the molecular retrosynthetic route using the above methods, and the above methods require determining a maximum exploration height in the early stage of building a molecular retrosynthetic tree. Consequently, if the maximum exploration height is too small, it is difficult to complete the construction of molecular retrosynthetic trees within a limited height for complex molecules. On the contrary, if the maximum exploration height is too large, the time required will increase exponentially, resulting in low efficiency and low accuracy of molecular retrosynthetic route design.

### SUMMARY

Embodiments of this disclosure provide a training method and apparatus for a neural network for determining a molecular retrosynthetic route, a method for determining a molecular retrosynthetic route, an apparatus, a device, and a readable storage medium.

In an embodiment, a training method for a neural network configured to determine a molecular retrosynthetic route includes determining first disassembly paths of a plurality of first molecules such that a first disassembly path is determined for each of the plurality of first molecules based on molecular expression information of the respective one of the plurality of first molecules. The method also includes obtaining a first cost dictionary based on the first disassembly paths of the first molecules, the first cost dictionary comprising the molecular expression information of each of the first molecules and cost value information corresponding to each of the first molecules. The cost value information of each first molecule represents a cost required to disassemble the respective first molecule according to the first disassembly path of the respective first molecule. The method also includes determining molecular expression information of second molecules based on the first disassembly paths of the first molecules, each of the second molecules being a molecule that is obtained by disassembling a corresponding first molecule based on the first disassembly path of the corresponding first molecule. Each of the second molecules is capable of being further disassembled. The method also includes determining a plurality of third molecules from the second molecules, each of the third molecules representing a class of the second molecules, and obtaining a second cost dictionary based on second disassembly paths of the third molecules. The second cost dictionary includes molecular expression information of each of the third molecules and cost value information corresponding to each of the third molecules, wherein the cost value information of each third molecule represents a cost required to disassemble the respective third molecule according to the second disassembly path of the respective third molecule. The method also includes performing training based on the first cost dictionary and the second cost dictionary to obtain a target neural network, the target neural network being configured to output cost value information corresponding to a target molecule according to input molecular expression information of the target molecule. The cost value information corresponding to the target molecule is used for synthesizing a retrosynthetic route for the target molecule.

In an embodiment, a method for determining a molecular retrosynthetic route includes receiving molecular expression information of a target molecule, the molecular expression information representing a three-dimensional chemical structure of the target molecule. The method also includes inputting the molecular expression information of the target molecule into a neural network for determining a molecular retrosynthetic route, and determining a disassembly path of the target molecule based on the neural network. The determined disassembly path is a disassembly path with a minimum disassembly cost among at least one possible disassembly path of the target molecule. The method also includes obtaining molecular retrosynthetic route information of the target molecule based on the determined disassembly path.

In an embodiment, a training apparatus for a neural network includes processing circuitry configured to determine first disassembly paths of a plurality of first molecules such that a first disassembly path is determined for each of the plurality of first molecules based on molecular expression information of the respective one of the plurality of first molecules. The processing circuitry is further configured to obtain a first cost dictionary based on the first disassembly paths of the first molecules. The first cost dictionary includes the molecular expression information of each of the first molecules and cost value information corresponding to each of the first molecules, wherein the cost value information of each first molecule represents a cost required to disassemble the respective first molecule according to the first disassembly path of the respective first molecule. The processing circuitry is further configured to determine molecular expression information of second molecules based on the first disassembly paths of the first molecules, each of the second molecules being a molecule that is obtained by disassembling a corresponding first molecule based on the first disassembly path of the corresponding first molecule. Each of the second molecules is capable of being further disassembled. The processing circuitry is further configured to determine a plurality of third molecules from the second molecules, each of the third molecules representing a class of the second molecules, and obtain a second cost dictionary based on second disassembly paths of the third molecules. The second cost dictionary includes molecular expression information of each of the third molecules and cost value information corresponding to each of the third molecules, wherein the cost value information of each third molecule represents a cost required to disassemble the respective third molecule according to the second disassembly path of the respective third molecule. The processing circuitry is further configured to perform training based on the first cost dictionary and the second cost dictionary to obtain a target neural network. The target neural network is configured to output cost value information corresponding to a target molecule according to input molecular expression information of the target molecule, the cost value information corresponding to the target molecule being used for synthesizing a retrosynthetic route for the target molecule.

Details of one or more embodiments of this disclosure are provided in the accompanying drawings and descriptions below. Based on the specification, the accompanying drawings, and the claims of this disclosure, other features, objectives, and advantages of this disclosure become clearer.

### BRIEF DESCRIPTION OF THE DRAWINGS

To describe technical solutions in embodiments of this disclosure, the following briefly introduces accompanying drawings describing the embodiments. The accompanying drawings in the following description show merely some embodiments of this disclosure, and a person of ordinary skill in the art may still derive other accompanying drawings according to the accompanying drawings.

FIG. 1 is a schematic diagram of an implementation environment of a training method for a neural network for determining a molecular retrosynthetic route according to an embodiment of this disclosure.

FIG. 2 is an architecture diagram of building a molecular retrosynthetic tree based on a hierarchical manner according to an embodiment of this disclosure.

FIG. 3 is a flowchart of a training method for a neural network for determining a molecular retrosynthetic route according to an embodiment of this disclosure.

FIG. 4 is a flowchart of another training method for a neural network for determining a molecular retrosynthetic route according to an embodiment of this disclosure.

FIG. 5 is a flowchart of a method for obtaining a second cost vocabulary according to an embodiment of this disclosure.

FIG. 6 is an architecture diagram of a training method for a neural network for determining a molecular retrosynthetic route according to an embodiment of this disclosure.

FIG. 7 is a flowchart of a method for determining a molecular retrosynthetic route according to an embodiment of this disclosure.

FIG. 8 is a block diagram of a training apparatus for a neural network for determining a molecular retrosynthetic route according to an embodiment of this disclosure.

FIG. 9 is a schematic structural diagram of a server according to an embodiment of this disclosure.

### DESCRIPTION OF EMBODIMENTS

To make objectives, technical solutions, and advantages of this disclosure clearer, the following further describes implementations of this disclosure in detail with reference to the accompanying drawings. Exemplary embodiments are described in detail herein, and examples thereof are shown in the accompanying drawings. When the following descriptions are made with reference to the accompanying drawings, unless otherwise indicated, the same numbers in different accompanying drawings represent the same or similar elements. The following implementations described in the following exemplary embodiments do not represent all implementations that are consistent with this disclosure. Instead, they are merely examples of apparatuses and methods consistent with aspects related to this disclosure as recited in the appended claims. The following briefly introduces technologies and terms that may be used in the embodiments of this disclosure.

Artificial Intelligence (AI) is a theory, method, technology, and application system that uses a digital computer or a machine controlled by the digital computer to simulate, extend, and expand human intelligence, perceive an environment, acquire knowledge, and use knowledge to obtain an optimal result. In other words, AI is a comprehensive technology in computer science. This technology attempts to understand the essence of intelligence and produce a new intelligent machine that can react in a manner similar to human intelligence. AI is to study the design principles and implementation methods of various intelligent machines, so that the machines can perceive, infer, and make decisions.

Machine learning (ML) is a multi-field interdiscipline and relates to a plurality of disciplines such as probability theory, statistics, approximation theory, convex analysis, and algorithm complexity theory. ML specializes in studying how a computer simulates or implements a human learning behavior to acquire new knowledge or skills, and reorganize an existing knowledge structure, so as to keep improving its performance. ML is the core of AI, is a basic way to make a computer intelligent, and is applied to various fields of AI. ML and deep learning generally include technologies such as an artificial neural network, a belief network, reinforcement learning, transfer learning, inductive learning, and learning from demonstrations.

A Markov decision process (MDP) is a mathematical model of sequential decision and is used for simulating a random strategy and returns that the intelligent agent can implement in an environment in which a system state has a Markov property. The MDP is constructed based on a set of interactive objects, that is, the intelligent agent and the environment, including elements such as states, actions, strategies, and rewards. In simulation of the MDP, the intelligent agent perceives the current system state and implements actions on the environment according to the strategy, thereby changing the state of the environment and obtaining awards. The accumulation of awards over time is referred to as rewards.

The molecular expression information is information for representing a three-dimensional chemical structure of a molecule. For example, the molecular expression information is a simplified molecular input line entry specification (SMILES) of the molecule, that is, the chemical structure of the molecule is represented by a character string. For another example, the molecular expression information is a molecular graph used for representing the molecular structure, as shown in Table 1. Table 1 shows the properties of nodes and edges commonly used in the molecular graph.

TABLE 1

| Category | Property | Description |
|---|---|---|
| Node | Atom type | Carbon, nitrogen, oxygen, hydrogen, |
| | | fluorine, sulfur, chlorine, |
| | | etc. (one-hot encoding) |
| | Atomic number | Number of protons (integer) |
| | Positive valence | Receive electron (binary) |
| | Negative valence | Contributed electron (binary) |
| | Aromatic | In aromatic (binary) |
| | Hybridization | sp, sp2, sp3 (one-hot encoding |
| | | or zero) |
| | Number of hydrogen | (integer) |
| | atoms | |
| Edge | Chemical bond type | Single bond, double bond, |
| | | triple bond, aromatic |
| | | (one-hot encoding) |
| | Atomic distance | (real number) |

A Jaccard similarity coefficient is used to compare the similarities and differences between finite sample sets. A larger value of the Jaccard similarity coefficient indicates a higher sample similarity. A Tanimoto coefficient is extended from the Jaccard coefficient, and is also known as a generalized Jaccard similarity coefficient.

A Synthetic Accessibility score (SA Score) is a method for quickly assessing the ease of synthesis of a large number of compounds based on the complexity of the molecules. In this method, based on the assumption that “substructures that frequently appear are easy to synthesize”, frequencies of extended-connectivity fingerprints of diameter 4 are weighted among 1 million compounds obtained from a bioactivity database of small organic molecules (PubChem), and the frequency of occurrence and molecular complexity are used as evaluation indicators to calculate the synthetic accessibility of the molecule. The value of the synthetic accessibility score is standardized as 1 (easy) to 10 (hard).

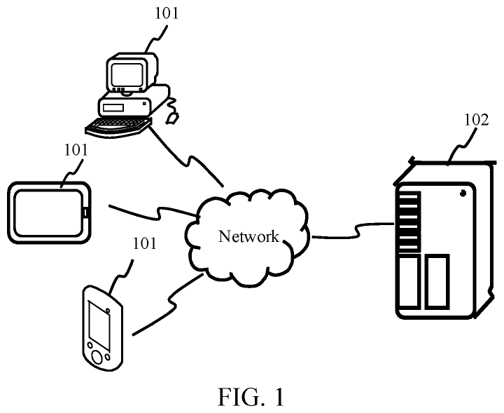

The following describes an implementation environment of the training method provided in the embodiments of this disclosure. FIG. 1 is a schematic diagram of an implementation environment of a training method for a neural network for determining a molecular retrosynthetic route according to an embodiment of this disclosure. The implementation environment includes: a terminal device 101 and a server 102.

The terminal 101 and the server 102 can be directly or indirectly connected in a wired or wireless communication manner, which is not limited in this disclosure. In one embodiment, the terminal 101 is a smartphone, a tablet computer, a laptop computer, a desktop computer, or the like, but is not limited thereto. The terminal 101 can provide the server 102 with basic information for determining a molecular retrosynthetic route, such as molecular expression information (including a molecular graph structure, simplified molecular input line entry specification, etc.), molecular cost value reference information (such as molecular synthetic accessibility score) and molecular retrosynthesis reference information (such as molecular disassembly scheme), etc.

The server 102 can be an independent physical server, or can be a server cluster including a plurality of physical servers or a distributed system, or can be a cloud server providing basic cloud computing services, such as a cloud service, a cloud database, cloud computing, a cloud function, cloud storage, a network service, cloud communication, a middleware service, a domain name service, a security service, a content delivery network (CDN), big data, and an artificial intelligence platform. The server 102 is configured to execute the method for determining a molecular retrosynthetic route provided in the embodiments of this disclosure, and to perform neural network training based on the basic information provided by the terminal 101. In one embodiment, the server 102 is capable of hosting a Linux operating system and GPU computing resources.

In one embodiment, in the process of training the neural network for determining a molecular retrosynthetic route, the server 102 is responsible for the main computing work, and the terminal 101 is responsible for the secondary computing work. Alternatively, the server 102 is responsible for secondary computing work, and the terminal 101 is responsible for primary computing work. or, the server 102 or the terminal 101 can be responsible for the computing work alone.

In one embodiment, the above training process adopts distributed training, for example, multiple computing nodes are respectively used for training. The server 102 includes a training server. The training server is a server cluster, including a plurality of servers serving as computing nodes. Each computing node performs a part of a training task respectively. A neural network model obtained through training can be transmitted to a target server to provide users with corresponding functions.

In one embodiment, the terminal 101 generally refers to one of a plurality of terminals. In this embodiment, the terminal 101 is merely used as an example for description. It can be understood by a person skilled in the art that there may be more terminals 101 and servers 1021. For example, there may be dozens of or hundreds of or more terminals 101. In this case, the implementation environment of the method for determining a molecular retrosynthetic route may further include other terminals. The quantity and the device type of the terminals are not limited in the embodiments of this disclosure.

In an embodiment, a standard communication technology and/or protocol is used for the foregoing wireless network or the wired network. The network is usually the Internet, but can alternatively be any other networks, including but not limited to a local area network (LAN), a metropolitan area network (MAN), a wide area network (WAN), a mobile, wired, or wireless network, or any combination of a dedicated network or a virtual dedicated network). In some embodiments, technologies and/or formats, such as hypertext markup language (HTML) and extensible markup language (XML), are used for representing data exchanged through a network. In addition, all or some links can be encrypted by using encryption technologies such as secure socket layer (SSL), transport layer security (TLS), virtual private network (VPN), and Internet Protocol security (IPsec). In other embodiments, custom and/or dedicated data communication technologies can also be used in place of or in addition to the foregoing data communication technologies.

In the embodiments of this disclosure, a method for determining a molecular retrosynthetic route is provided, which adopts hierarchical reinforcement learning. In the chemical field, retrosynthesis analysis is an important method for resolving a molecular synthetic route, and is also the simplest and most basic method for designing a molecular synthetic route. The essence lies in the decomposition of a target molecule. The structure of the target molecule is analyzed and gradually decomposed into simpler and easier-to-synthesize intermediates and raw materials, thereby completing the design of a molecular synthetic route. The intermediates refer to precursor compounds required for the synthesis of the target molecule, that is, organic compounds that are not readily available in the market and need to be synthesized. The raw materials refer to relatively simple organic compounds that are readily available in the market for synthesizing the target molecule. The exploration of a retrosynthetic route of a molecule is the process of constructing a retrosynthetic tree for the molecule. In the related art, training for the construction of a retrosynthetic tree of an existing molecule is generally performed based on a preset maximum exploration height. This method leads to the fact that if the maximum exploration height is too small, some more complex molecules are very difficult to perform. Consequently, if the maximum exploration height is too small, some relatively it is difficult to complete the construction of molecular retrosynthetic trees within a limited height for some for complex molecules. On the contrary, if the maximum exploration height is too large, the time required for construction will increase exponentially. In the training process for the construction of a molecular retrosynthetic tree in the embodiments of this disclosure, hierarchical construction is adopted, which can greatly reduce the large amount of calculation and time overheads required in the process of constructing a molecular retrosynthetic tree. For a detailed description, reference may be made to the following embodiments.

The following embodiments are all described using an example where the process of constructing a molecular retrosynthetic route is divided into two layers. However, the embodiments of this disclosure are not limited thereto, but the process of constructing a molecular retrosynthetic tree may be divided into multiple layers, which is not limited in the embodiments of this disclosure.

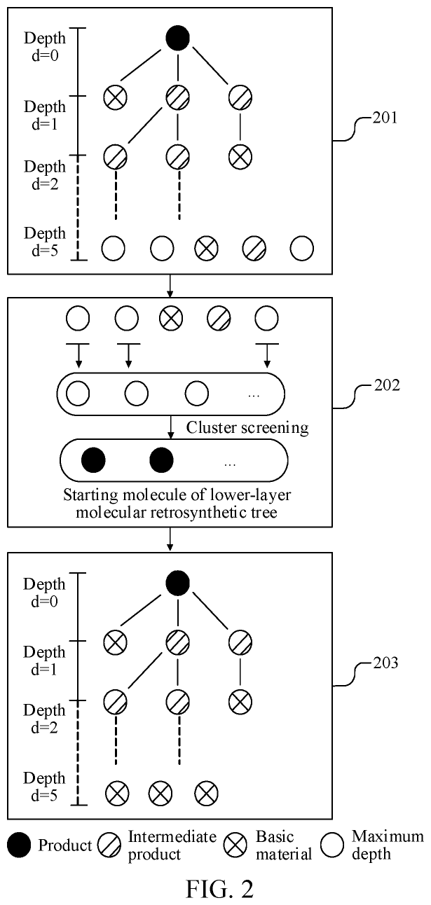

For example, referring to FIG. 2, FIG. 2 is an architecture diagram of building a molecular retrosynthetic tree based on a hierarchical manner according to an embodiment of this disclosure. As shown in FIG. 2, taking a maximum depth of the molecular retrosynthetic tree being 10 as an example, the construction of the entire molecular retrosynthetic tree is divided into upper (201) and lower (203) layers, and two smaller molecular retrosynthetic trees are used to replace the complete retrosynthetic reaction process. After the construction of the upper-layer molecular retrosynthetic tree is completed, a representative molecule is selected by molecular clustering (202) and screening and used as a starting molecule in the lower-layer molecular retrosynthetic tree, which effectively improves the exploration efficiency of the molecular retrosynthetic route, whereby accurate molecular cost information is more efficiently extracted.

A method for determining a molecular retrosynthetic route according to an embodiment of this disclosure is described in detail below.

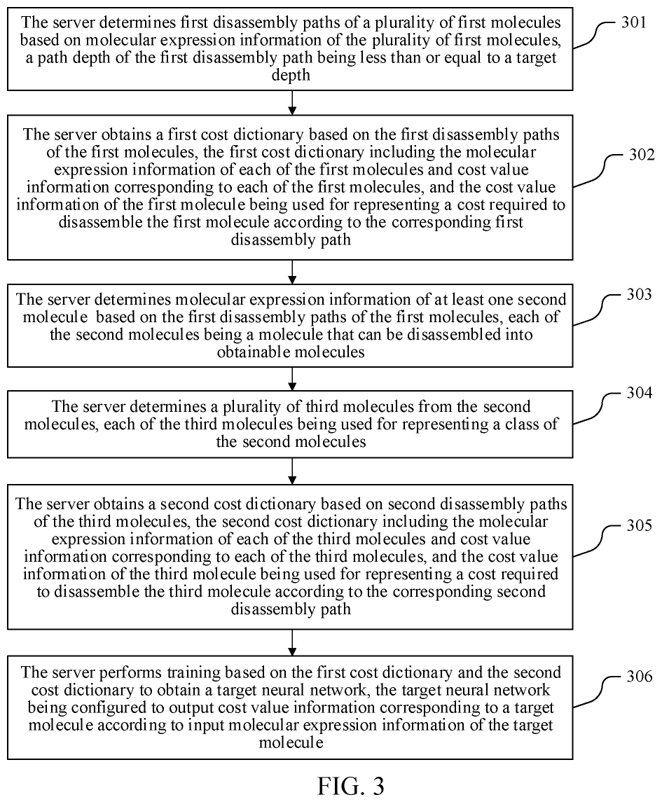

FIG. 3 is a flowchart of a method for determining a molecular retrosynthetic route according to an embodiment of this disclosure. As shown in FIG. 3, this embodiment of this disclosure is described using an example where the method is applied to a server. The method includes the following steps.

In step 301, the server determines first disassembly paths of a plurality of first molecules based on molecular expression information of the plurality of first molecules, a path depth of the first disassembly path being less than or equal to a target depth. For example, first disassembly paths of a plurality of first molecules are determined such that a first disassembly path is determined for each of the plurality of first molecules based on molecular expression information of the respective one of the plurality of first molecules.

In an embodiment of this disclosure, the first molecules are existing molecules. The molecular expression information is used for representing a three-dimensional chemical structure of a molecule. For example, the molecular expression information is a simplified molecular input line entry specification (SMILES) of the molecule, that is, the chemical structure of the molecule is represented by a character string. For another example, the molecular expression information is a molecular map used for representing the molecular structure, which is not limited in the embodiments of this disclosure. The first disassembly path refers to a path that requires the least cost to dissemble the first molecule until the target disassembly condition is met.

In step 302, the server obtains a first cost dictionary based on the first disassembly paths of the first molecules, the first cost dictionary including the molecular expression information of each of the first molecules and cost value information corresponding to each of the first molecules, and the cost value information of the first molecule being used for representing a cost required to disassemble the first molecule according to the corresponding first disassembly path.

For example, a first cost dictionary is obtained based on the first disassembly paths of the first molecules. The first cost dictionary includes the molecular expression information of each of the first molecules and cost value information corresponding to each of the first molecules, and the cost value information of each first molecule represents a cost required to disassemble the respective first molecule according to the first disassembly path of the respective first molecule.

In the embodiments of this disclosure, the molecular expression information and the corresponding cost value information in the first cost dictionary exist in a one-to-one correspondence manner.

In step 303, the server determines molecular expression information of at least one second molecule based on the first disassembly paths of the first molecules, each of the second molecules being a molecule that can be disassembled into obtainable molecules. For example, molecular expression information of second molecules is determined based on the first disassembly paths of the first molecules. Each of the second molecules is a molecule that is obtained by disassembling a corresponding first molecule based on the first disassembly path of the corresponding first molecule.

In the embodiments of this disclosure, after each first molecule is disassembled based on the corresponding first disassembly path, multiple molecules can be obtained. Among the multiple molecules, some molecules can be further disassembled, and there is a disassembly path. These molecules are determined as second molecules. That is, each of the second molecules is a molecule that can be disassembled into obtainable molecules among molecules obtained by disassembling the corresponding first molecule based on the first disassembly path.

In step 304, the server determines a plurality of third molecules from the second molecules, each of the third molecules being used for representing a class of the second molecules.

In the embodiments of this disclosure, the second molecules are divided into multiple sets based on structural similarities between molecules. The second molecules in each set have similar molecular structures. A third molecule is determined from each set. The third molecule is a representative molecule in the set to which the third molecule belongs.

In step 305, the server obtains a second cost dictionary based on second disassembly paths of the third molecules, the second cost dictionary including the molecular expression information of each of the third molecules and cost value information corresponding to each of the third molecules, and the cost value information of the third molecule being used for representing a cost required to disassemble the third molecule according to the corresponding second disassembly path. For example, a second cost dictionary is obtained based on second disassembly paths of the third molecules. The second cost dictionary includes molecular expression information of each of the third molecules and cost value information corresponding to each of the third molecules, and the cost value information of each third molecule represents a cost required to disassemble the respective third molecule according to the second disassembly path of the respective third molecule.

In the embodiments of this disclosure, the second disassembly path refers to a path that requires the least cost to dissemble the third molecule until the target disassembly condition is met. The molecular expression information and the corresponding cost value information in the second cost dictionary exist in a one-to-one correspondence manner.

In step 306, the server performs training based on the first cost dictionary and the second cost dictionary to obtain a target neural network, the target neural network being configured to output cost value information corresponding to a target molecule according to input molecular expression information of the target molecule.

It can be understood that the disassembly path corresponding to the cost value information corresponding to the target molecule is the disassembly path with the lowest disassembly cost value among all possible disassembly paths of the target molecule. Therefore, a retrosynthetic route for the target molecule may be synthesized based on the cost value information corresponding to the target molecule.

In one embodiment, the target molecule may be dissembled based on the disassembly path corresponding to the cost value information, and molecular retrosynthetic route information may be obtained according to a result of the disassembly.

In the embodiments of this disclosure, the server performs training based on the first cost dictionary to obtain a first neural network, performs training based on the second cost dictionary to obtain a second neural network, and finally combines the two neural networks to obtain the target neural network.

In the embodiments of this disclosure, through steps 301 to 306, after dividing the exploration of the retrosynthetic routes of multiple molecules into multiple layers, the server respectively obtains a cost dictionary corresponding to each layer according to an Lth layer and an (L+1)th layer obtained, where L is greater than or equal to 1. For each layer obtained in the exploration process, a corresponding cost dictionary can be obtained. Correspondingly, the server trains the neural network based on the first cost dictionary, the second cost dictionary and the cost dictionaries obtained by the first L-1 layers to obtain a trained neural network, so as to obtain multiple layers of molecular retrosynthetic routes of the molecule. In the embodiments of this disclosure, a training method for a neural network for determining a molecular retrosynthetic route is provided. When a retrosynthetic route of each of a plurality of molecules is determined, a concept of hierarchical learning is adopted. A training process of a molecular retrosynthetic route requiring deeper exploration is split into multiple layers for training to accelerate the training, and the complete retrosynthetic reaction process is replaced by multiple layers of molecular retrosynthetic routes. After the training of one layer of molecular retrosynthesis route is completed, a representative molecule is selected by molecular screening and used as a starting molecule in a next layer of molecular retrosynthetic route, which effectively improves the exploration efficiency of the molecular retrosynthetic route, whereby accurate molecular cost information is more efficiently extracted. The layered approach greatly reduces the computational overhead brought about by determining the molecular retrosynthetic route, and reduces the time for determining the molecular retrosynthetic route while the accuracy of the molecular retrosynthetic route is ensured.

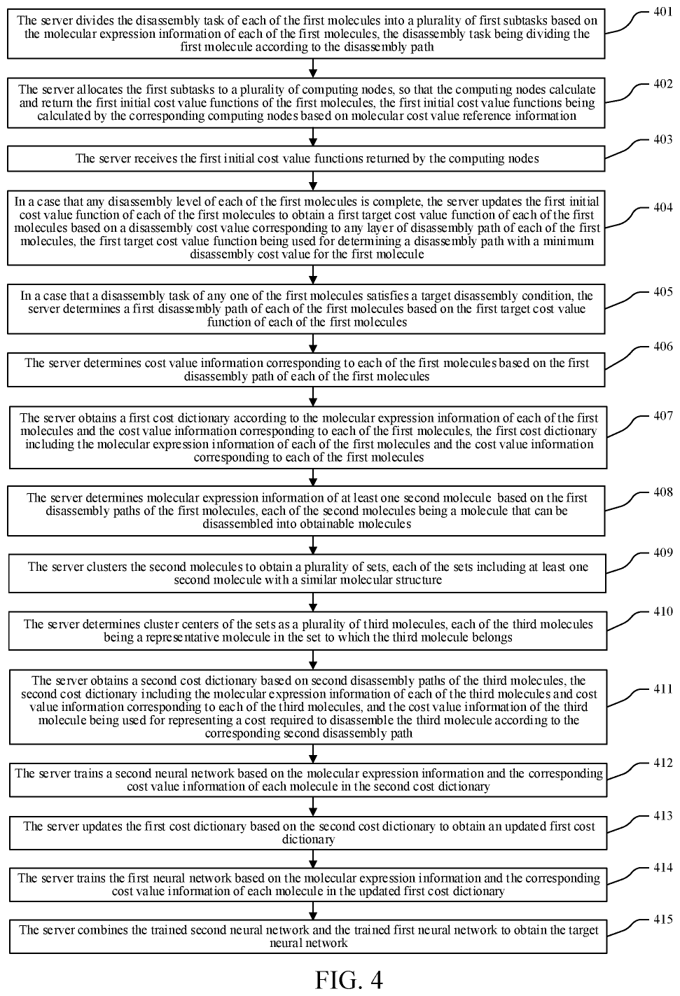

FIG. 4 is a flowchart of another training method for a neural network for determining a molecular retrosynthetic route according to an embodiment of this disclosure. As shown in FIG. 4, this embodiment of this disclosure is described using an example where the method is applied to a server and a molecular retrosynthetic route is divided into two layers. The method includes the following steps.

In step 401, the server divides the disassembly task of each of the first molecules into a plurality of first subtasks based on the molecular expression information of each of the first molecules, the disassembly task being dividing the first molecule according to the disassembly path.

In the embodiments of this disclosure, the disassembly task is to gradually dissemble a molecule into at least one simpler and easier-to-synthesize molecule according to the disassembly path through the analysis of the molecular structure of the molecule based on molecular expression information of the molecule.

In an implementation, the server is associated with a molecule database, where the molecule database is used to store molecular expression information of existing molecules. The server can extract the molecular expression information of each first molecule from the molecule database, generate a disassembly task of each first molecule based on the molecular expression information of each first molecule, and then divide the disassembly task of each first molecule into multiple first subtasks. Each first subtask includes the disassembly task of at least one first molecule.

On the premise that one first molecule corresponds to one disassembly task, the division of the disassembly tasks of the multiple first molecules by the server includes any one of the following implementations.

In a first implementation, the server performs an average division according to a current quantity of first molecules and a current quantity of computing nodes. For example, the current quantity of first molecules is divided by the current quantity of computing nodes, and the obtained value is used to determine the quantity of first subtasks in each computing node. Each first subtask includes the same quantity of disassembly tasks of first molecules.

In a second implementation, the server divides the disassembly tasks by levels of complexity of chemical three-dimensional structures of molecules according to the molecular expression information of the first molecules, for example, allocates a disassembly task of the first molecule with a single benzene ring into a first subtask, and allocates a disassembly task of the first molecule with more than one benzene ring into another first subtask, and so on. Levels of complexity of chemical three-dimensional structures of the first molecules corresponding to one first subtask are similar.

In a third implementation, the server divides the disassembly tasks according to the current quantity of computing nodes and computing capabilities of the computing nodes. For example, when a quantity of subtasks currently running on a computing node is greater than a threshold, the disassembly tasks of 10 first molecule are allocated to one first subtask; when the quantity of subtasks currently running on a computing node is less than the threshold, the disassembly tasks of 100 first molecules are allocated to another first subtask; and so on. Each first subtask includes a different quantity of disassembly tasks of first molecules.

The manner of dividing the disassembly tasks of the plurality of first molecules is not specifically limited in the embodiments of this disclosure. Any of the methods provided above may be adopted, or any of the methods may be combined to obtain a more complex division manner.

In step 402, the server allocates the first subtasks to a plurality of computing nodes, so that the computing nodes calculate and return the first initial cost value functions of the first molecules, the first initial cost value functions being calculated by the corresponding computing nodes based on molecular cost value reference information.

In the embodiments of this disclosure, the server can process data based on multiple computing nodes at the same time. The server assigns multiple first subtasks to multiple computing nodes, with one computing node being responsible for the disassembly task of at least one first molecule. The parallel computing mode using multiple computing nodes can accelerate the training process and improve the training speed. The first initial cost value function is an initial cost value function of each first molecule calculated by the computing node based on the molecular expression information of each first molecule and by using the molecular cost value reference information. The molecular cost value reference information is used for representing synthetic accessibility of the molecule. The cost value function is used for determining the disassembly path with the least cost to disassemble the molecule according to the molecular expression information of the molecule. In the initial stage of disassembly of each first molecule, the cost value function of each first molecule is initialized using the molecular cost value reference information, to avoids the unstable strategy that is likely to be generated due to cost value functions obtained by random initialization, thereby improving the stability of the strategy for determining a molecular disassembly route based on cost value functions.

In an implementation, the molecular cost value reference information is a synthetic accessibility score, and the server initializes the cost value function of the molecule by using the synthetic accessibility score. By using the priori advantage of the synthetic accessibility score in representing the ease of synthesis of the molecule, the convergence speed of the molecular cost value function can be improved to a certain extent.

In an implementation, when the server allocates the first subtasks to multiple computing nodes, any one of the following manners may be adopted.

In a first implementation, the quantity of the first subtasks is the same as the current quantity of computing nodes, and the server allocates the first subtasks to the computing nodes in a one-to-one correspondence.

In a second implementation, the quantity of the first subtasks is greater than the current quantity of computing nodes, and the server allocates the first subtasks to the computing nodes according to current computing capabilities of the computing nodes. For example, more than one first subtask is allocated to a computing node with strong computing power, while only one first subtask is allocated to a computing node with weak computing power.

In a third implementation, the server allocates the first subtasks according to the quantity of disassembly tasks of first molecules in the first subtask and computing power of the computing nodes. For example, the first subtask including the disassembly tasks of 100 first molecules is assigned to the computing node with strong computing power, and the first subtask including the disassembly tasks of 10 first molecules is assigned to the computing node with weak computing power.

The manner in which the first subtasks are assigned to multiple computing nodes is not specifically limited in the embodiments of this disclosure. Any of the methods provided above may be adopted, or any of the methods may be combined to obtain a more complex allocation manner.

In step 403, the server receives the first initial cost value functions returned by the computing nodes.

In the embodiments of this disclosure, based on the first initial cost value functions calculated by the computing nodes in the above step 402, the first initial cost value functions corresponding to the first molecules returned by the computing nodes are received.

Steps 401 to 403 are an implementation of the parallel molecular retrosynthetic route exploration process based on a distributed training framework provided in the embodiments of this disclosure. The distributed computing method can speed up the calculation while maintaining the original computing effect, thereby increasing the training speed. In another implementation, the server executes the disassembly tasks of the first molecules independently, not based on a distributed training framework. For example, the server obtains the first initial cost value functions of the first molecules based on the molecular expression information of the first molecules and the molecular cost value reference information, which is not specifically limited in the embodiments of this disclosure.

In step 404, in a case that any disassembly level of each of the first molecules is complete, the server updates the first initial cost value function of each of the first molecules to obtain a first target cost value function of each of the first molecules based on a disassembly cost value corresponding to any layer of disassembly path of each of the first molecules, the first target cost value function being used for determining a disassembly path with a minimum disassembly cost value for the first molecule.

In the embodiments of this disclosure, when the disassembly tasks of the first molecules are executed, a complete disassembly path of a first molecule consists of at least one disassembly level. One disassembly level is used to perform one step of disassembly on a first molecule, that is, the disassembly path of a first molecule consists of at least one step of disassembly.

There are various disassembly methods for each step of disassembly. Each disassembly method corresponds to a disassembly cost value. The disassembly cost value is used for representing the cost required to disassemble the first molecule to a current level based on the current disassembly method. When any disassembly level of each first molecule is complete, the server obtains at least one disassembly cost value based on at least one disassembly method existing in the any disassembly level. The server updates the first initial cost value function of each of the first molecules based on the at least one disassembly cost value to obtain a first target cost value function of each of the first molecules based on a disassembly cost value corresponding to any layer of disassembly path of each of the first molecules, the first target cost value function being used for determining a disassembly path with a minimum disassembly cost value for the first molecule.

For example, when the server disassembles the first molecules, there are three disassembly methods A₁, A₂, and A₃ for a first molecule at a first disassembly level A₁, that is, when a first step of disassembly is performed on the first molecule. The three disassembly methods correspond to disassembly cost values a₁, a₂, and a₃ respectively. Based on the three disassembly cost values, a first initial cost value function of the first molecule is updated, and it is determined according to the updated cost value function that when the disassembly method A₁ is used for assembly, the corresponding disassembly cost value al is the smallest. Therefore, the server performs a second disassembly level B on the basis of the disassembly method A₁ at the first disassembly level A, that is, performs a second step of disassembly. Similarly, there are three disassembly methods B₁, B₂, and B₃ at the second disassembly level B. The three disassembly methods correspond to disassembly cost values b₁, b₂, and b₃ respectively. The cost value function having been updated based on the disassembly level A is further updated based on these three disassembly cost values, to determine the disassembly method with the smallest disassembly cost value at the disassembly level B. The server sequentially performs a next disassembly level according to the above method, and when any disassembly level is complete, iteratively updates the first initial cost value function to finally obtain the first target cost value function.

In an implementation, a constraint condition for determining the disassembly path with the smallest disassembly cost value is called a strategy. When performing the disassembly task for each first molecule, formula (1) is used to calculate a disassembly cost value required by a disassembly path obtained after at least one disassembly level is completed on the first molecule based on this strategy. In this case, different disassembly paths can be obtained based on different disassembly methods, i.e., different disassembly cost values can be obtained. When any disassembly level of the first molecule is complete, a disassembly cost value based on at least one disassembly path can be obtained. Based on the calculated at least one disassembly cost value, the first initial cost value function of the first molecule is updated to obtain a first target cost value function v_{*}(m) shown in formula (2). In one embodiment, after the first target cost value function is obtained, the strategy is iteratively updated according to formula (3). π(r|m) is used as a strategy to disassemble molecules, that is, the disassembly paths of the first molecules may be generated based on this strategy. This strategy is iteratively updated to obtain an updated strategy π′(r|m). The cost required when the first molecule is disassembled according to the updated strategy π′(r|m) is the smallest. Formulas (1) to (3) are as follows:

cₜₒₜ=Σᵣcᵣₓₙ(r) (1)

In formula (1), r represents a reaction that can be selected, that is, a disassembly method existing in the disassembly path of the first molecule, cₜₒₜ represents the total disassembly cost required to disassemble the first molecule, and crxn represents the cost value when the first molecule is disassembled in a disassembly level according to the reaction r.

$${v}_{*} ( m ) = {min}_{r} [ {c}_{r \times n} ( r ) + {\sum }_{{m}^{′} ∈ M ( r )} {v}_{*} ( {m}^{′} ) ] \qquad ( 2 )$$

In formula (2), m represents the product, that is, the first molecule, and M(r) represents a set of molecules.

$${π}^{′} ( r \vert m ) = \{ \begin{matrix}1 if r = arg {min}_{r ∈ ℛ ( m )} [ {c}_{r \times n} ( r ) + {\sum }_{{m}^{′} ∈ ℳ ( r )} {v}_{π} ( {m}^{′} ) ] \\ 0 otherwise\end{matrix} \qquad ( 3 )$$

In formula (3), v_{π}(m′) represents a cost value function for expanding the first molecule according to π to obtain the product m.

In an implementation, when the server performs the disassembly task for each first molecule, there are K disassembly levels, where K is greater than or equal to 1. Each time a disassembly level is completed, the corresponding disassembly cost value is 1. When a molecule obtained after disassembly is performed according to at least one disassembly method existing in any disassembly level is a molecule existing in the molecule database, the disassembly cost value of the current disassembly method is 0. When the molecule obtained is an impossible molecule, for example, when the molecule does not exist in the molecule database, the disassembly cost value of the current disassembly method is 100. The server calculates the disassembly cost value of each first molecule based on any disassembly level based on the above formula (1). Finally, the server can update the first initial cost value function of the first molecule based on the disassembly cost value obtained through any disassembly level, so as to obtain the first target cost value function.

In an implementation, the server performs the disassembly task for each first molecule based on the molecular retrosynthesis reference information. The molecular retrosynthesis reference information is used for providing any disassembly level of each first molecule with at least one disassembly method based on the disassembly level. For example, the molecular retrosynthesis reference information is a computer-aided retrosynthesis method based on molecular similarity. This method can provide available disassembly schemes for molecules. For another example, the molecular retrosynthesis reference information is a template neural network, which is used to provide at least one reaction template, that is, a disassembly method, for the disassembly of the molecule. The reaction template is used to describe the process of a type of chemical reaction, including the breaking of an existing chemical bond and the formation of a new chemical bond.

In an implementation, in the above process of disassembling each first molecule, the disassembling process of each first molecule is modeled as an expanding game. Using π(r|m) as a strategy for disassembling the molecules, where m represents the product and r represents a reaction that can be selected, the cost value function of the current molecule can be obtained. During the expansion process, the first target cost value function v_{*}(m) is continuously updated to determine the disassembly path with the smallest disassembly cost. The strategy is iteratively updated at each round of expansion.

In step 405, in a case that a disassembly task of any one of the first molecules satisfies a target disassembly condition, the server determines a first disassembly path of each of the first molecules based on the first target cost value function of each of the first molecules.

In the embodiments of this disclosure, the target disassembly condition means that a path depth of a disassembly path obtained for each first molecule based on at least one disassembly level is less than a target depth and there is no disassembly method for the obtained molecules, or means that the path depth of the disassembly path obtained for each first molecule based on at least one disassembly level is equal to the target depth. The target depth is a depth threshold preset by the server. For example, the depth threshold is set to 5.

The following is an example where the target depth is 5.

If a path depth of a disassembly path obtained after P disassembly levels are performed for a first molecule is less than 5, where P is greater than or equal to 1 and less than 5, and there is no further disassembly method for the obtained molecules, that is, the obtained molecules are all molecules that can be obtained from the molecule database, then the disassembly task of the first molecule satisfies the target disassembly condition, and the server obtains a first target cost value function of the first molecule based on the current disassembly level, and determines a disassembly path with the smallest disassembly cost value based on this function, where the disassembly path is the first disassembly path of the first molecule.

If a path depth of a disassembly path obtained after Q disassembly levels are performed for a first molecule, where Q is equal to 5, the disassembly task of the first molecule satisfies the target disassembly condition, and the server obtains a first target cost value function of the first molecule based on the current disassembly level, and determines a disassembly path with the smallest disassembly cost value based on this function, where the disassembly path is the first disassembly path of the first molecule.

In step 406, the server determines cost value information corresponding to each of the first molecules based on the first disassembly path of each of the first molecules.

In the embodiments of this disclosure, the cost value information of each first molecule is used for representing a cost required to disassemble the first molecule according to the corresponding first disassembly path. In an implementation, the server determines the cost value information corresponding to each first molecule based on the first target cost value function of each first molecule, that is, v_{*}(m). In another implementation, during disassembly of each first molecule, each time any disassembly level is completed, a disassembly path with the minimum disassembly cost value based on the current disassembly level can be obtained, that is, the cost value information corresponding to each first molecule based on the current disassembly level can be obtained.

In step 407, the server obtains a first cost dictionary according to the molecular expression information of each of the first molecules and the cost value information corresponding to each of the first molecules, the first cost dictionary including the molecular expression information of each of the first molecules and the cost value information corresponding to each of the first molecules.

In the embodiments of this disclosure, the molecular expression information and the corresponding cost value information in the first cost dictionary exist in a one-to-one correspondence manner. For example, a key-value pair may be used in the first cost dictionary to represent the molecular expression information of each molecule and the cost value information corresponding to each molecule. That is, there are N key-value pairs in the first cost dictionary, where N is greater than or equal to 1: {Smile1:cost1}; {Smile2:cost2} . . . {SmileN:costN}, which are respectively used for representing molecule 1 to molecule N and cost value information corresponding to the molecules.

By performing the above steps 401 to 407, the server completes the exploration of the upper-layer retrosynthetic routes of multiple first molecules, and obtains a first cost dictionary corresponding to the upper-layer retrosynthetic routes.

408. The server determines molecular expression information of at least one second molecule based on the first disassembly paths of the first molecules, each of the second molecules being a molecule that can be disassembled into obtainable molecules.

In the embodiments of this disclosure, after each first molecule is disassembled based on the corresponding first disassembly path, multiple molecules can be obtained. Among the multiple molecules, some molecules can be further disassembled, and there is a disassembly path. These molecules are determined as second molecules.

In an implementation, when performing a disassembly task corresponding to a certain first molecule, the server disassembles the first molecule based on the corresponding first disassembly path to obtain at least one molecule, and then determines molecular expression information of the obtained at least one molecule. Based on this, the server determines whether the obtained molecules can be further disassembled. If one of the molecules can be further disassembled, it is determined whether there is a disassembly method for this molecule, and this molecule is determined as a second molecule.

In the embodiments of this disclosure, according to the sequence of the above steps 406 to 408, the server first determines the cost value information of each first molecule based on the first disassembly path of each first molecule to obtain the first cost dictionary, and then determines at least one second molecule. In another implementation, the server may first determine at least one second molecule based on the first disassembly path of each first molecule according to step 408, and then execute steps 406 and 407 to obtain the first cost dictionary. This is not specifically limited in the embodiments of this disclosure.

In step 409, the server clusters the second molecules to obtain a plurality of sets, each of the sets including at least one second molecule with a similar molecular structure.

In the embodiments of this disclosure, clustering means a process of grouping the second molecules into multiple sets based on structural similarities between the molecules. The second molecules in each set have similar molecular structures. In one embodiment, the server is capable of classifying the at least one second molecule based on other classification methods, e.g., a Bayesian classification algorithm or the like. This is not specifically limited in the embodiments of this disclosure.

In an implementation, the server clusters the second molecules based on a Taylor Butina (TB) algorithm. The structural similarity between two second molecules is determined by a Tanimoto coefficient, as shown in formula (4):

$$T = \frac{{m}_{i} \cdot {m}_{j}}{{\vert {m}_{i} \vert}^{2} + {\vert {m}_{j} \vert}^{2} - {m}_{i} \cdot {m}_{j}} \qquad ( 4 )$$

In formula (4), mᵢ represents an ECPF4 molecular fingerprint of molecule i, and mᵢ represents an ECPF4 molecular fingerprint of molecule j. For example, the ECPF4 molecular fingerprint has a fingerprint length of 1024 and a radius of 3.

When the Tanimoto coefficient between two molecules is less than a preset threshold, it is determined that the two molecules belong to the same class of molecules, and the two molecules are put into the same set. For example, the preset threshold is 0.4. The setting of the preset threshold is not specifically limited in the embodiments of this disclosure and may be adjusted according to actual situations.

In the embodiments of this disclosure, the at least one second molecule is clustered using the TB algorithm. In another implementation, the server can cluster the second molecules using other clustering algorithms, for example, k-means clustering algorithm, mean-shift clustering algorithm, etc. This is not specifically limited in the embodiments of this disclosure.

In step 410, the server determines cluster centers of the sets as a plurality of third molecules, each of the third molecules being a representative molecule in the set to which the third molecule belongs.

In the embodiments of this disclosure, the cluster centers are the centers of the plurality of sets generated by clustering the second molecules, the center is a representative second molecule in the corresponding set, and the representative second molecule is determined as the third molecule.

After exploring the upper-layer retrosynthetic routes of the first molecules by performing the above steps 408 to 410, the server screens a plurality of obtained molecules that can be further decomposed and for which there is still a disassembly method, to obtain a plurality of representative third molecules. These third molecules are used as starting molecules of lower-layer retrosynthetic routes, so that during the exploration of the retrosynthetic route of each molecule, it is not necessary to continuously perform disassembly until the maximum depth is reached or until the exploration of the entire route is completed, thereby saving time. In the process of layering, the use of representative molecules as the third molecules greatly reduces the calculation amount of the subsequent training process, reduces the time for determining a molecular retrosynthetic route, and further improves the training speed.

411. The server obtains a second cost dictionary based on second disassembly paths of the third molecules, the second cost dictionary including the molecular expression information of each of the third molecules and cost value information corresponding to each of the third molecules, and the cost value information of the third molecule being used for representing a cost required to disassemble the third molecule according to the corresponding second disassembly path.

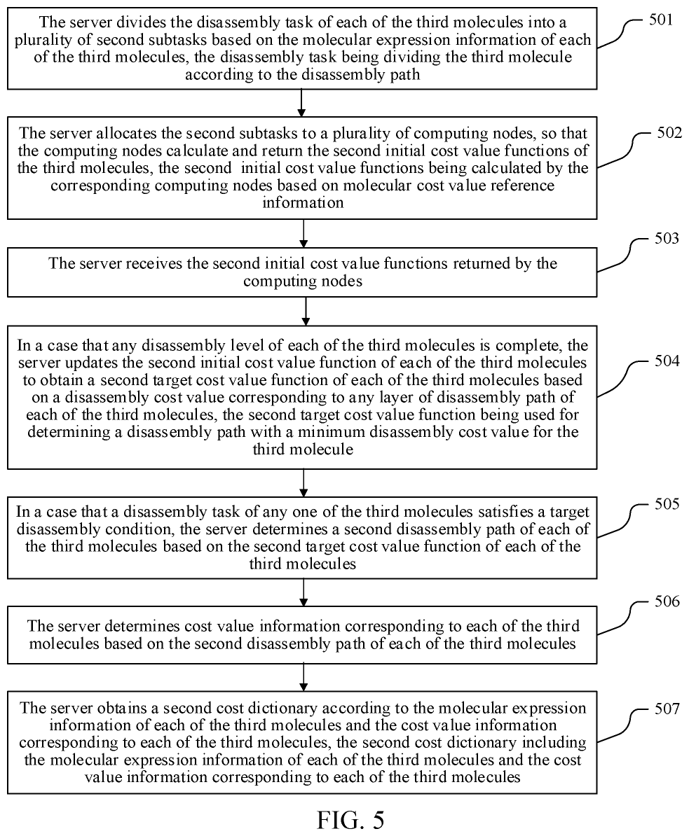

In the embodiments of this disclosure, reference is made to FIG. 5 for the process of obtaining the second cost dictionary based on the second disassembly paths of the third molecules. FIG. 5 is a flowchart of a method for obtaining a second cost vocabulary according to an embodiment of this disclosure. Specifically, the method includes the following steps 501 to 507. Steps 501 to 507 are similar to the process of executing the above steps 401 to 407, so steps 501 to 507 are merely briefly described below, and the specific implementations will not be repeated.

In step 501, the server divides the disassembly task of each of the third molecules into a plurality of second subtasks based on the molecular expression information of each of the third molecules, the disassembly task being dividing the third molecule according to the disassembly path.

In the embodiments of this disclosure, the third molecules are molecules obtained after performing the above step 410. The server divides the disassembly tasks of the plurality of third molecules into multiple subtasks based on the molecular expression information of each third molecule.

In step 502, the server allocates the second subtasks to a plurality of computing nodes, so that the computing nodes calculate and return the second initial cost value functions of the third molecules, the second initial cost value functions being calculated by the corresponding computing nodes based on molecular cost value reference information.

In the embodiments of this disclosure, the server can process data based on multiple computing nodes at the same time. The server assigns multiple second subtasks to multiple computing nodes, with one second computing node being responsible for the disassembly task of at least one third molecule. The second initial cost value function is an initial cost value function of each third molecule calculated by the computing node based on the molecular expression information of each third molecule and by using the molecular cost value reference information. The cost value function is used for determining the disassembly path with the least cost to disassemble the molecule according to the molecular expression information of the molecule. In the initial stage of disassembly of each third molecule, the cost value function of each third molecule is initialized using the molecular cost value reference information, to avoids the unstable strategy that is likely to be generated due to cost value functions obtained by random initialization, thereby improving the stability of the strategy for determining a molecular disassembly route based on cost value functions.

In step 503, the server receives the second initial cost value functions returned by the computing nodes.

In the embodiments of this disclosure, based on the second initial cost value functions calculated by the computing nodes in the above step 502, the server can receive the second initial cost value functions corresponding to the third molecules returned by the computing nodes.

In step 504, in a case that any disassembly level of each of the third molecules is complete, the server updates the second initial cost value function of each of the third molecules to obtain a second target cost value function of each of the third molecules based on a disassembly cost value corresponding to any layer of disassembly path of each of the third molecules, the second target cost value function being used for determining a disassembly path with a minimum disassembly cost value for the third molecule.

In the embodiments of this disclosure, when the disassembly tasks of the third molecules are executed, a complete disassembly path of a third molecule consists of at least one disassembly level. One disassembly level is used to perform one step of disassembly on a third molecule, that is, the disassembly path of a third molecule consists of at least one step of disassembly.

There are various disassembly methods for each step of disassembly. Each disassembly method corresponds to a disassembly cost value. The disassembly cost value is used for representing the cost required to disassemble the third molecule to a current level based on the current disassembly method. When any disassembly level of each third molecule is complete, the server obtains at least one disassembly cost value based on at least one disassembly method existing in the any disassembly level. The server updates the second initial cost value function of each of the third molecules based on the at least one disassembly cost value to obtain a second target cost value function of each of the third molecules based on a disassembly cost value corresponding to any layer of disassembly path of each of the third molecules, the second target cost value function being used for determining a disassembly path with a minimum disassembly cost value for the third molecule.

In step 505, in a case that a disassembly task of any one of the third molecules satisfies a target disassembly condition, the server determines a second disassembly path of each of the third molecules based on the second target cost value function of each of the third molecules.

In the embodiments of this disclosure, the target disassembly condition means that a path depth of a disassembly path obtained for each third molecule based on at least one disassembly level is less than a target depth and there is no disassembly method for the obtained molecules, or means that the path depth of the disassembly path obtained for each third molecule based on at least one disassembly level is equal to the target depth. The target depth is a depth threshold preset by the server. For example, the depth threshold is set to 5. The second disassembly path refers to a path that requires the least cost to dissemble the third molecule until the target disassembly condition is met.

506. The server determines cost value information corresponding to each of the third molecules based on the second disassembly path of each of the third molecules.

In the embodiments of this disclosure, the cost value information of each third molecule is used for representing a cost required to disassemble the third molecule according to the corresponding second disassembly path.

In step 507, the server obtains a second cost dictionary according to the molecular expression information of each of the third molecules and the cost value information corresponding to each of the third molecules, the second cost dictionary including the molecular expression information of each of the third molecules and the cost value information corresponding to each of the third molecules

In the embodiments of this disclosure, the molecular expression information and the corresponding cost value information in the second cost dictionary exist in a one-to-one correspondence manner. For example, a key-value pair may be used in the second cost dictionary to represent the molecular expression information of each molecule and the cost value information corresponding to each molecule. That is, there are M key-value pairs in the second cost dictionary, where M is greater than or equal to 1: {Smilei:costi}; {Smile2:cost2} . . . {Smilem:costm}, which are respectively used for representing molecule 1 to molecule M and cost value information corresponding to the molecules.

By performing the above steps 501 to 507, the server completes the exploration of the lower-layer retrosynthetic routes, and obtains a second cost dictionary corresponding to the lower-layer retrosynthetic routes. By such a layering method, the exploration of the entire retrosynthetic routes of multiple first molecules is layered. The exploration of the upper-layer retrosynthetic routes is completed first, and after representative molecules are selected, the exploration of the lower-layer retrosynthetic routes is completed. Whereby, the time required for exploring molecular retrosynthetic routes is greatly reduced.

In step 412, the server trains a second neural network based on the molecular expression information and the corresponding cost value information of each molecule in the second cost dictionary.

In the embodiments of this disclosure, the neural network includes a first neural network and a second neural network. The first neural network is a neural network obtained by training based on information in the first cost dictionary. The second neural network is a neural network obtained by training based on information in the second cost dictionary. Step 412 includes the following steps: the server inputs the molecular expression information of a molecule in the second cost dictionary into the second neural network, and performs calculation based on a network parameter of the second neural network to obtain predicted cost value information corresponding to the molecule; the server determines a model loss of the second neural network based on the predicted cost value information corresponding to each molecule and the cost value information corresponding to each molecule in the second cost dictionary; and the server adjusts the network parameter in the second neural network according to the model loss of the second neural network, inputs the molecular expression information of a new molecule again based on the adjusted second neural network, and iteratively adjusts the network parameter in the second neural network according to the predicted cost value information obtained by each input and the cost value information corresponding to the inputted molecule, until the model loss of the second neural network meets a target condition; then the server determines the current second neural network as the trained second neural network.

The process of training the second neural network in the embodiments of this disclosure may further include other steps, which will not be detailed here.

In step 413, the server updates the first cost dictionary based on the second cost dictionary to obtain an updated first cost dictionary.

In the embodiments of this disclosure, the third molecules in the second cost dictionary are determined by clustering the plurality of molecules obtained by disassembling the first molecule in the first cost dictionary. The second cost dictionary includes the molecule expression information of each third molecule and the cost value information corresponding to each third molecule. The server uses the molecule expression information of each third molecule and the cost value information corresponding to each third molecule to update the cost value information of each first molecule, to obtain the updated first cost dictionary. Through the bottom-up update process of the cost value information of each first molecule by the server, the accuracy of determining the cost of molecular retrosynthesis is improved.

In an implementation, the server updates the first cost dictionary based on the second cost dictionary by the following specific process: determining the molecular expression information of the first molecules respectively corresponding to the third molecule according to the molecular expression information of the third molecules, and then summing up the cost value information of each third molecule in the second cost dictionary and the cost value information of the corresponding first molecule in the first cost dictionary to obtain updated cost value information of the first molecule, that is, to obtain the updated first cost dictionary. For example, according to the cost value information of a third molecule, the server obtains that the minimum disassembly cost for disassembling the third molecule is 10, and the minimum disassembly cost of the corresponding first molecule is 20. In this case, the updated minimum disassembly cost of the first molecule is 30. The manner of updating the first cost dictionary is not limited in the embodiments of this disclosure.

In step 414, the server trains the first neural network based on the molecular expression information and the corresponding cost value information of each molecule in the updated first cost dictionary.

In an embodiment of this disclosure, step 414 includes the following steps: the server inputs the molecular expression information of a molecule in the first cost dictionary into the first neural network, and performs calculation based on a network parameter of the first neural network to obtain predicted cost value information corresponding to the molecule; the server determines a model loss of the first neural network based on the predicted cost value information corresponding to each molecule and the cost value information corresponding to each molecule in the first cost dictionary; and the server adjusts the network parameter in the first neural network according to the model loss of the first neural network, inputs the molecular expression information of a new molecule again based on the adjusted first neural network, and iteratively adjusts the network parameter in the first neural network according to the predicted cost value information obtained by each input and the cost value information corresponding to the inputted molecule, until the model loss of the first neural network meets a target condition; then the server determines the current first neural network as the trained first neural network.

The process of training the first neural network in the embodiments of this disclosure may further include other steps, which will not be detailed here.

In step 415, the server combines the trained second neural network and the trained first neural network to obtain the target neural network.

In the embodiments of this disclosure, the target neural network is configured to output cost value information corresponding to a target molecule according to input molecular expression information of the target molecule. The server combines the trained second neural network and the trained first neural network to obtain the target neural network. For example, the server obtains the target neural network in a serial manner. For the target molecule, the server inputs the molecular expression information of the target molecule into the first neural network, and outputs the cost value information of the target molecule based on the upper-layer retrosynthetic route. Then, the server determines molecules for which there is a further disassembly method among molecules obtained after exploration of the upper-layer retrosynthetic route of the target molecule, inputs the molecules for which there is a further disassembly method into the second neural network, outputs the cost value information of the target molecule based on the lower-layer retrosynthetic route, and finally obtains the complete retrosynthetic route of the target molecule. The manner of obtaining the target neural network is not specifically limited in the embodiments of this disclosure.

By performing the above steps 412 to 415, the server performs training based on the second cost dictionary and the updated first cost dictionary to obtain the target neural network. This can maximize the generalization ability of the target neural network of this disclosure for molecules that do not participate in the training process, so that a disassembly path can be obtained for any molecule based on the target neural network.

In the embodiments of this disclosure, according to the sequence of the above steps 412 to 415, the server first trains the second neural network, then updates the first cost dictionary, and trains the first neural network. In another implementation, the server may first execute step 413 to update the first cost dictionary, and then execute steps 412, 414, and 415 to obtain the target neural network. This is not specifically limited in the embodiments of this disclosure.

In the embodiments of this disclosure, the above steps 412 to 415 are an implementation of the process of obtaining the target neural network through training according to the embodiments of this disclosure. In an embodiment, the server can also obtain the target neural network through other training methods, which is not specifically limited in the embodiments of this disclosure.

In addition, referring to Table 2, Table 2 shows a comparison of experimental effects of related methods and the embodiments of this disclosure. In the experiment, the time required for successfully decomposing the same number of molecules based on a standard data set is obtained. In contrast, compared with the related methods, the method provided in the embodiments of this disclosure requires a significantly shorter time in decomposing the same number of molecules than those required by the other methods.

TABLE 2

| | Number of molecules | | |
|---|---|---|---|
| | successfully decomposed | | |
| Algorithm | 1000 | 2000 | 3000 |
| Monte Carlo Tree (MCTS) | 55 h | 98 h | — |
| Reinforcement learning | 52 h | 107 h | 146 h |
| Distributed | 23 h | 45 h | 63 h |
| reinforcement learning | | | |
| Embodiment of | 12 h | 25 h | 35 h |
| this disclosure | | | |

Referring to Table 3, Table 3 shows a comparison of experimental effects of related methods and the embodiments of this disclosure. In the experiment, a decomposition result of decomposing the same number of molecules based on a standard data set is obtained. The experimental results show that compared with related methods, when decomposing the same number of molecules, the method provided in the embodiments of this disclosure can successfully decompose a larger number of molecules, with a smaller number of molecules failing to be decomposed and a smaller number of failures due to an excessively large number of decomposition layers.

TABLE 3

| | Decomposition Result | | |
|---|---|---|---|
| | | | Number of |
| | | | failures |
| | | | due to |
| | | Number of | excessively |
| | | failures | large |
| | Number of | due to | number of |
| | successful | inability to | decomposition |
| Algorithm | decompositions | decompose | layers |
| Monte Carlo | 2567 | 574 | 698 |
| Tree (MCTS) | | | |
| Reinforcement | 3122 | 194 | 523 |
| learning | | | |
| Distributed | 3085 | 289 | 465 |
| reinforcement | | | |
| learning | | | |
| Embodiment of | 3578 | 65 | 196 |
| this disclosure | | | |

Certainly, the above different implementations may be combined with each other to form different embodiments, which is not limited in the embodiments of this disclosure.

In the embodiments of this disclosure, a training method for a neural network for determining a molecular retrosynthetic route is provided. When a retrosynthetic route of each of a plurality of molecules is determined, a concept of hierarchical learning is adopted. A training process of a molecular retrosynthetic route requiring deeper exploration is split into multiple layers for training to accelerate the training, and the complete retrosynthetic reaction process is replaced by multiple layers of molecular retrosynthetic routes. After the training of one layer of molecular retrosynthesis route is completed, a representative molecule is selected by molecular screening and used as a starting molecule in a next layer of molecular retrosynthetic route, which effectively improves the exploration efficiency of the molecular retrosynthetic route, whereby accurate molecular cost information is more efficiently extracted. The hierarchical approach greatly reduces the computational overhead brought about by determining the molecular retrosynthetic route, and reduces the time for determining the molecular retrosynthetic route while ensuring the accuracy of the molecular retrosynthetic route.

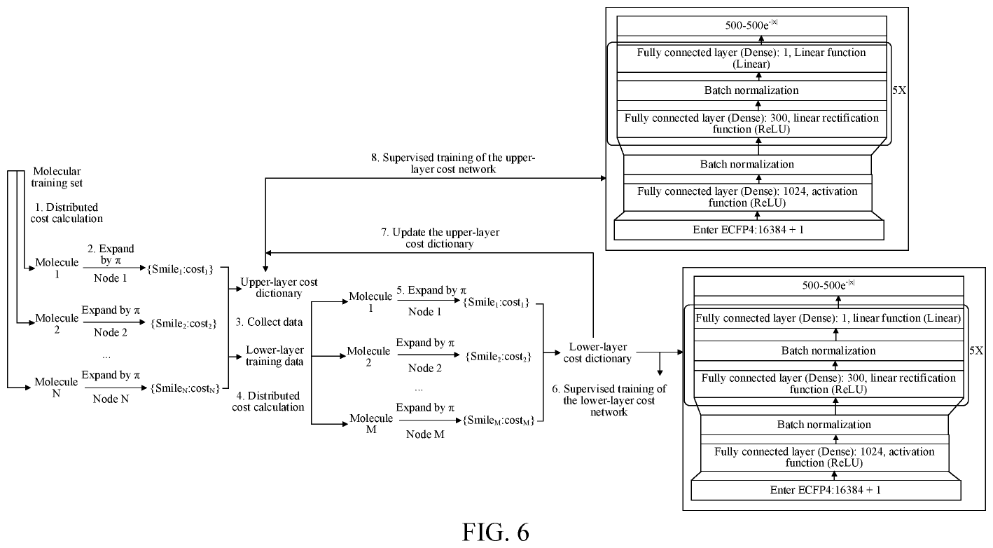

In the following, an example of a training method for a neural network for determining a molecular retrosynthetic route provided in the embodiments of this disclosure is described with reference to an actual situation. As shown in FIG. 6, FIG. 6 is an architecture diagram of a training method for a neural network for determining a molecular retrosynthetic route according to an embodiment of this disclosure. An example where the process of constructing a molecular retrosynthetic route is divided into two layers is described.

First, for molecules in a molecular training set, a distributed training framework is used to perform parallel exploration of upper-layer retrosynthetic routes, and all molecular disassembly tasks are allocated to N computing nodes, where N is greater than or equal to 1. The computing nodes perform the disassembly tasks of the molecules respectively. In the early stage of disassembly of each molecule, the cost value function is initialized using a molecular synthetic accessibility score. By using the priori advantage of the synthetic accessibility score in representing the ease of synthesis of the molecule, the convergence speed of the molecular cost value function is improved. Then according to the strategy π(r|m), each molecule is expanded, and finally the cost of each molecule under the corresponding strategy is obtained. When the calculation performed by each computing node is complete, the molecular expression information and cost value information of all molecules are summarized to obtain an upper-layer cost dictionary.

Secondly, after the exploration of the upper-layer retrosynthetic routes is complete, molecules that can be further decomposed and for which there is still a disassembly method among the obtained molecules are collected, and clustered using the TB algorithm. Representative molecules are selected as an initial training set for the exploration of lower-layer molecular retrosynthetic routes, that is, as lower-layer training data. The molecules in the lower-layer molecular training set are expanded by a molecule expanding process similar to that for the upper layer to obtain a lower-layer cost dictionary.

Thirdly, the upper-layer cost dictionary is updated based on the lower-layer cost dictionary, the corresponding neural networks are trained based on the two cost dictionaries respectively, and supervised training of the two neural networks is performed using deep learning technology to obtain the target neural network that can explore retrosynthetic routes of new target molecules. The process of supervised training of the neural network is as follows: Molecular expression information of a molecule is inputted, and processed by a first fully connected layer (Dense), followed by batch normalization processing of the data. Then the data is processed by a second fully connected layer, followed by batch normalization processing. The process of processing by a second fully connected layer followed by batch normalization processing of the data is repeated five times. Finally, 500-500e^{-|x|} is outputted through a third fully connected layer, where x is a variable.

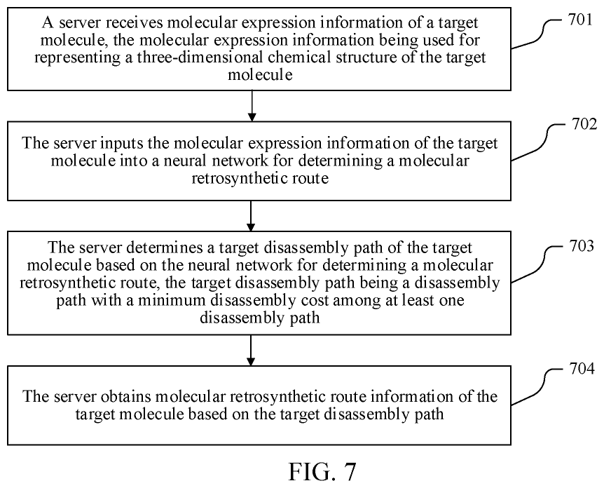

FIG. 7 is a flowchart of a method for determining a molecular retrosynthetic route according to an embodiment of this disclosure. As shown in FIG. 7, this embodiment of this disclosure is described using an example where the method is applied to a server. The method includes the following steps.

In step 701, a server receives molecular expression information of a target molecule, the molecular expression information being used for representing a three-dimensional chemical structure of the target molecule.

In the embodiments of this disclosure, the target molecule is a molecule that cannot be obtained from a molecule database. The server receives the molecular expression information of the target molecule. For example, the server receives a simplified molecular input line entry specification of the target molecule, that is, a string of characters used for representing a three-dimensional chemical structure of the molecule.

In step 702, the server inputs the molecular expression information of the target molecule into a neural network for determining a molecular retrosynthetic route.

In the embodiments of this disclosure, after receiving the molecular expression information of the target molecule, the server inputs same into the neural network for determining a molecular retrosynthetic route that is provided in the embodiments of this disclosure.

In step 703, the server determines a target disassembly path of the target molecule based on the neural network for determining a molecular retrosynthetic route, the target disassembly path being a disassembly path with a minimum disassembly cost among at least one disassembly path. For example, a disassembly path of the target molecule is determined based on the neural network, the determined disassembly path being a disassembly path with a minimum disassembly cost among at least one possible disassembly path of the target molecule.

In the embodiments of this disclosure, based on the neural network for determining a molecular retrosynthetic route that is provided in the embodiments of this disclosure, cost value information corresponding to the target molecule is outputted. A disassembly path corresponding to the cost value information is a disassembly path with the lowest disassembly cost value among all possible disassembly paths of the target molecule.

In an implementation, step 703 includes the following steps: the neural network for determining a molecular retrosynthetic route including a first neural network and a second neural network, outputting cost value information of an upper-layer retrosynthetic route of the target molecule based on the first neural network; determining a first disassembly path of the target molecule, a path depth of the first disassembly path being less than or equal to a target depth; determining molecular expression information of a molecule for which a further disassembly method exists among molecules obtained by disassembling the target molecule based on the first disassembly path; inputting the molecular expression information of the molecule for which a further disassembly method exists into the second neural network; outputting cost value information of a lower-layer retrosynthetic route of the target molecule based on the second neural network; determining a second disassembly path of the target molecule; and determining the target disassembly path of the target molecule based on the first disassembly path and the second disassembly path.

In step 704, the server obtains molecular retrosynthetic route information of the target molecule based on the target disassembly path.

In the embodiments of this disclosure, according to the target disassembly path obtained in step 703, a disassembly reaction of the target molecule based on each step of the disassembly path is obtained, and a complete retrosynthetic route of the target molecule is finally determined.

In the embodiments of this disclosure, a method for determining a molecular retrosynthetic route is provided. Through a neural network for determining a molecular retrosynthetic route, the retrosynthetic route of a target molecule is obtained, with a short time and high accuracy.

Although the steps in the flowcharts of the embodiments are displayed sequentially according to instructions of arrows, these steps are not necessarily performed sequentially according to a sequence instructed by the arrows. Unless otherwise clearly specified in this specification, the steps are performed without any strict sequence limit, and may be performed in other sequences. In addition, at least some steps in the flowcharts in the foregoing embodiments may include a plurality of steps or a plurality of stages. The steps or the stages are not necessarily performed at the same moment, but may be performed at different moments. The steps or the stages are not necessarily performed in sequence, but may be performed in turn or alternately with another step or at least some of steps or stages of the another step.

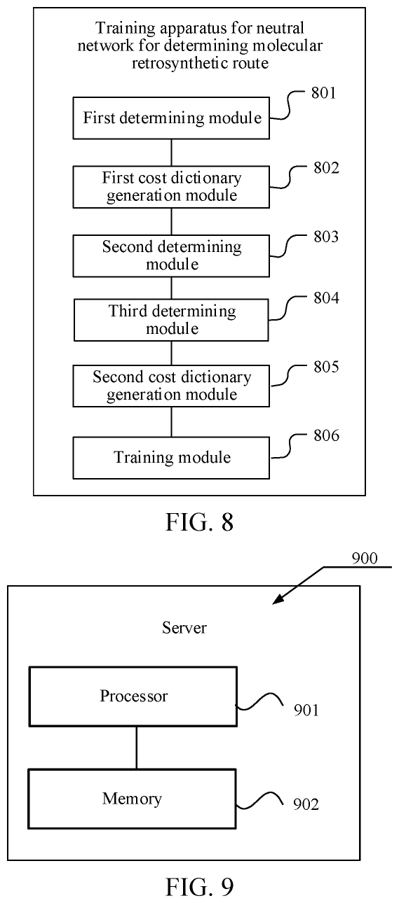

FIG. 8 is a block diagram of a training apparatus for a neural network for determining a molecular retrosynthetic route according to an embodiment of this disclosure. The apparatus is configured to execute the steps of the training method for a neural network for determining a molecular retrosynthetic route. Referring to FIG. 8, the apparatus includes: a first determining module 801, a first cost dictionary generation module 802, a second determining module 803, a third determining module 804, a second cost dictionary generation module 805, and a training module 806.

The first determining module 801 is configured to determine first disassembly paths of a plurality of first molecules based on molecular expression information of the plurality of first molecules, a path depth of the first disassembly path being less than or equal to a target depth.

The first cost dictionary generation module 802 is configured to obtain a first cost dictionary based on the first disassembly paths of the first molecules, the first cost dictionary including the molecular expression information of each of the first molecules and cost value information corresponding to each of the first molecules, and the cost value information of the first molecule being used for representing a cost required to disassemble the first molecule according to the corresponding first disassembly path.

The second determining module 803 is configured to determine molecular expression information of at least one second molecule based on the first disassembly paths of the first molecules, each of the second molecules being a molecule that can be disassembled into obtainable molecules among molecules obtained by disassembling the corresponding first molecule based on the first disassembly path.

The third determining module 804 is configured to determine a plurality of third molecules from the second molecules, each of the third molecules being used for representing a class of the second molecules.

The second cost dictionary generation module 805 is configured to obtain a second cost dictionary based on second disassembly paths of the third molecules, the second cost dictionary including the molecular expression information of each of the third molecules and cost value information corresponding to each of the third molecules, and the cost value information of the third molecule being used for representing a cost required to disassemble the third molecule according to the corresponding second disassembly path.

The training module 806 is configured to perform training based on the first cost dictionary and the second cost dictionary to obtain a target neural network, the target neural network being configured to output cost value information corresponding to a target molecule according to input molecular expression information of the target molecule.

In an implementation, the first determining module 801 includes: an obtaining unit, configured to obtain a first initial cost value function of each of the first molecules based on the molecular expression information and cost value reference information of each of the first molecules; an updating unit, configured to, in a case that any disassembly level of each of the first molecules is complete, update the first initial cost value function of each of the first molecules to obtain a first target cost value function of each of the first molecules based on a disassembly cost value corresponding to any layer of disassembly path of each of the first molecules, the first target cost value function being used for determining a disassembly path with a minimum disassembly cost value for the first molecule; and a determining unit, configured to, in a case that a disassembly task of any one of the first molecules satisfies a target disassembly condition, determine a first disassembly path of each of the first molecules based on the first target cost value function of each of the first molecules.

In an implementation, the obtaining unit is configured to execute operations of: dividing the disassembly task of each of the first molecules into a plurality of first subtasks based on the molecular expression information of each of the first molecules, the disassembly task being dividing the first molecule according to the disassembly path; allocating the first subtasks to a plurality of computing nodes, so that the computing nodes calculate and return the first initial cost value functions of the first molecules, the first initial cost value functions being calculated by the corresponding computing nodes based on molecular cost value reference information, and the molecular cost value reference information being used for representing synthetic accessibility of the molecule; and receiving the first initial cost value functions returned by the computing nodes.

In an implementation, the first cost dictionary generation module 802 is configured to execute operations of: determining cost value information corresponding to each of the first molecules based on the first disassembly path of each of the first molecules; and obtaining the first cost dictionary according to the molecular expression information of each of the first molecules and the cost value information corresponding to each of the first molecules.

In an implementation, the third determining module 804 is configured to execute operations of: clustering the second molecules to obtain a plurality of sets, each of the sets including at least one second molecule with a similar molecular structure; and obtaining the plurality of third molecules by respectively determining a cluster center of each of the sets as the third molecule corresponding to the set, each of the third molecules being a representative molecule in the set to which the third molecule belongs.

In an implementation, the training module 806 includes: a first training unit, configured to train a second neural network based on the molecular expression information and the corresponding cost value information of each molecule in the second cost dictionary; a second cost dictionary updating unit, configured to update the first cost dictionary based on the second cost dictionary to obtain an updated first cost dictionary; a second training unit, configured to train the first neural network based on the molecular expression information and the corresponding cost value information of each molecule in the updated first cost dictionary; and a combining unit, configured to combine the trained second neural network and the trained first neural network to obtain the target neural network.

In an implementation, the first training unit is configured to execute operations of: inputting the molecular expression information of each molecule in the second cost dictionary into the second neural network to obtain predicted cost value information corresponding to each molecule; determining a model loss of the second neural network based on the predicted cost value information corresponding to each molecule and the cost value information corresponding to each molecule in the second cost dictionary; and adjusting a network parameter in the second neural network according to the model loss of the second neural network.

In the embodiments of this disclosure, a training apparatus for a neural network for determining a molecular retrosynthetic route is provided. When a retrosynthetic route of each of a plurality of molecules is determined, a concept of hierarchical learning is adopted. A training process of a molecular retrosynthetic route requiring deeper exploration is split into multiple layers for training to accelerate the training, and the complete retrosynthetic reaction process is replaced by multiple layers of molecular retrosynthetic routes. After the training of one layer of molecular retrosynthesis route is completed, a representative molecule is selected by molecular screening and used as a starting molecule in a next layer of molecular retrosynthetic route, which effectively improves the exploration efficiency of the molecular retrosynthetic route, whereby accurate molecular cost information is more efficiently extracted. The layered approach greatly reduces the computational overhead brought about by determining the molecular retrosynthetic route, and reduces the time for determining the molecular retrosynthetic route while the accuracy of the molecular retrosynthetic route is ensured.

According to an embodiment, an apparatus for determining a molecular retrosynthetic route is also provided. The apparatus includes: a receiving module, configured to receive molecular expression information of a target molecule, the molecular expression information being used for representing a three-dimensional chemical structure of the target molecule; an input module, configured to input the molecular expression information of the target molecule into a neural network for determining a molecular retrosynthetic route; a disassembly path determining module, configured to determine a target disassembly path of the target molecule based on the neural network for determining a molecular retrosynthetic route, the target disassembly path being a disassembly path with a minimum disassembly cost among at least one disassembly path; and a route determining module, configured to obtain molecular retrosynthetic route information of the target molecule based on the target disassembly path.

In one embodiment, the neural network for determining a molecular retrosynthetic route includes a first neural network and a second neural network; and the disassembly path determining module is further configured to execute operations of: outputting cost value information of an upper-layer retrosynthetic route of the target molecule based on the first neural network; determining a first disassembly path of the target molecule based on the cost value information of the upper-layer retrosynthetic route of the target molecule; determining molecular expression information of a molecule for which a further disassembly method exists among molecules obtained by disassembling the target molecule based on the first disassembly path; inputting the molecular expression information of the molecule for which a further disassembly method exists into the second neural network; outputting cost value information of a lower-layer retrosynthetic route of the target molecule based on the second neural network; determining a second disassembly path of the target molecule based on the cost value information of the lower-layer retrosynthetic route of the target molecule; and determining the target disassembly path of the target molecule based on the first disassembly path and the second disassembly path.

The apparatus provided in the foregoing embodiments is described by using division into the foregoing functional modules as an example. In actual applications, the foregoing functions may be allocated to and completed by different functional modules according to requirements, that is, the internal structure of the apparatus is divided into different functional modules, to complete all or some of the foregoing described functions. In addition, the apparatus and the corresponding method embodiments provided in the foregoing embodiments belong to the same concept. For the specific implementation process, reference may be made to the method embodiments, and details are not described herein again.

When the computer device is configured as a server, FIG. 9 is a schematic structural diagram of a server according to embodiment of this disclosure. The server 900 may vary greatly due to different configurations or different performance, and can include one or more central processing units (CPUs) 901 (including processing circuitry) and one or more memories 902 (including a non-transitory computer-readable storage medium). The memory 902 stores at least one computer-readable instruction, the at least one computer-readable instruction being loaded and executed by the one or more processors 901 to implement the training method for a neural network for determining a molecular retrosynthetic route and the method for determining a molecular retrosynthetic route that are provided in the foregoing method embodiments. certainly, the server 900 may also have a wired or wireless network interface, a keyboard, an input/output interface and other components to facilitate input/output. The server 900 may also include other components for implementing device functions. Details are not described herein.

The embodiments of this disclosure further provide one or more computer-readable storage media. The one or more computer-readable storage media are applicable to a computer device. The one or more computer-readable storage media store at least one computer-readable instruction. The at least one computer-readable instruction is loaded and executed by one or more processors to implement the operations executed by a computer device in the methods of the above embodiments.

An embodiment of this disclosure further provides a computer program product, including computer instructions, the computer instructions being stored in a computer-readable storage medium. One or more processors of the computer device reads the computer-readable instructions from the computer-readable storage medium, and the one or more processors execute the computer-readable instructions to cause the computer device to perform the steps in the methods provided in the above implementations.

In an embodiment, a non-transitory computer-readable storage medium stores computer-readable instructions which, when executed by a computer device, cause the computer device to perform a training method for a neural network configured to determine a molecular retrosynthetic route. The training method includes determining first disassembly paths of a plurality of first molecules such that a first disassembly path is determined for each of the plurality of first molecules based on molecular expression information of the respective one of the plurality of first molecules. The method also includes obtaining a first cost dictionary based on the first disassembly paths of the first molecules, the first cost dictionary comprising the molecular expression information of each of the first molecules and cost value information corresponding to each of the first molecules. The cost value information of each first molecule represents a cost required to disassemble the respective first molecule according to the first disassembly path of the respective first molecule. The method also includes determining molecular expression information of second molecules based on the first disassembly paths of the first molecules, each of the second molecules being a molecule that is obtained by disassembling a corresponding first molecule based on the first disassembly path of the corresponding first molecule. The method also includes determining a plurality of third molecules from the second molecules, each of the third molecules representing a class of the second molecules, and obtaining a second cost dictionary based on second disassembly paths of the third molecules. The second cost dictionary includes molecular expression information of each of the third molecules and cost value information corresponding to each of the third molecules, wherein the cost value information of each third molecule represents a cost required to disassemble the respective third molecule according to the second disassembly path of the respective third molecule. The method also includes performing training based on the first cost dictionary and the second cost dictionary to obtain a target neural network, the target neural network being configured to output cost value information corresponding to a target molecule according to input molecular expression information of the target molecule. The cost value information corresponding to the target molecule is used for synthesizing a retrosynthetic route for the target molecule.

In an embodiment, a non-transitory computer-readable storage medium stores computer-readable instructions which, when executed by a computer device, cause the computer device to perform a method for determining a molecular retrosynthetic route. The method includes receiving molecular expression information of a target molecule, the molecular expression information representing a three-dimensional chemical structure of the target molecule. The method also includes inputting the molecular expression information of the target molecule into a neural network for determining a molecular retrosynthetic route, and determining a disassembly path of the target molecule based on the neural network. The determined disassembly path is a disassembly path with a minimum disassembly cost among at least one possible disassembly path of the target molecule. The method also includes obtaining molecular retrosynthetic route information of the target molecule based on the determined disassembly path.

A person of ordinary skill in the art may understand that all or some of the steps of the embodiments may be implemented by hardware or a computer-readable instruction instructing related hardware. The computer-readable instruction may be stored in one or more computer-readable storage media. The storage medium may be an ROM, a magnetic disk, an optical disc, or the like.

The term module (and other similar terms such as unit, submodule, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module.

The foregoing disclosure includes some exemplary embodiments of this disclosure which are not intended to limit the scope of this disclosure. Other embodiments shall also fall within the scope of this disclosure.

What is claimed is:

1. A training method for a neural network configured to determine a molecular retrosynthetic route, the method comprising:
determining first disassembly paths of a plurality of first molecules such that a first disassembly path is determined for each of the plurality of first molecules based on molecular expression information of the respective one of the plurality of first molecules;
obtaining a first cost dictionary based on the first disassembly paths of the first molecules, the first cost dictionary comprising the molecular expression information of each of the first molecules and cost value information corresponding to each of the first molecules, wherein the cost value information of each first molecule represents a cost required to disassemble the respective first molecule according to the first disassembly path of the respective first molecule;
determining molecular expression information of second molecules based on the first disassembly paths of the first molecules, each of the second molecules being a molecule that is obtained by disassembling a corresponding first molecule based on the first disassembly path of the corresponding first molecule, wherein each of the second molecules is capable of being further disassembled;
determining a plurality of third molecules from the second molecules, each of the third molecules representing a class of the second molecules;
obtaining a second cost dictionary based on second disassembly paths of the third molecules, the second cost dictionary comprising molecular expression information of each of the third molecules and cost value information corresponding to each of the third molecules, wherein the cost value information of each third molecule represents a cost required to disassemble the respective third molecule according to the second disassembly path of the respective third molecule; and
performing training based on the first cost dictionary and the second cost dictionary to obtain a target neural network, the target neural network being configured to output cost value information corresponding to a target molecule according to input molecular expression information of the target molecule, the cost value information corresponding to the target molecule being used for synthesizing a retrosynthetic route for the target molecule.

2. The method according to claim 1, wherein the determining the first disassembly paths comprises:
obtaining an initial cost value function of each of the first molecules based on the molecular expression information and cost value reference information of the respective first molecule;
in response to a determination that a disassembly level of each of the first molecules is complete, updating the initial cost value function of the respective first molecule to obtain a cost value function of the respective first molecule based on a disassembly cost value corresponding to the disassembly level of the respective first molecule, the cost value function being used for determining a disassembly path with a minimum disassembly cost value for the respective first molecule; and
in response to a determination that a disassembly task of one of the first molecules satisfies a disassembly condition, determining a first disassembly path of the one of the first molecules based on the cost value function of the one of the first molecules.

3. The method according to claim 2, wherein the disassembly condition is satisfied when there is no disassembly method for a molecule obtained by the disassembly task of the one of the first molecules, or when a path depth of the disassembly task obtained for the one of the first molecules is equal to a preset depth.

4. The method according to claim 2, wherein the obtaining the initial cost function comprises:
dividing a disassembly task of each of the first molecules into a plurality of first subtasks based on the molecular expression information of the respective first molecule, the disassembly task dividing the respective first molecule according to the disassembly path;
allocating the first subtasks to a plurality of computing nodes, so that the computing nodes calculate and return the initial cost value function of the respective first molecule, the initial cost value function being calculated by the computing nodes based on molecular cost value reference information representing synthetic accessibility of the respective first molecule; and
receiving the initial cost value functions returned by the computing nodes for each of the first molecules.

5. The method according to claim 1, wherein the obtaining the first cost dictionary comprises:
determining the cost value information corresponding to each of the first molecules based on the first disassembly path of each of the first molecules; and
obtaining the first cost dictionary according to the molecular expression information of each of the first molecules and the cost value information corresponding to each of the first molecules.

6. The method according to claim 1, wherein the determining the plurality of third molecules comprises:
clustering the second molecules to obtain a plurality of sets, each of the sets comprising one or more second molecules with a related molecular structure; and
obtaining the plurality of third molecules by respectively determining a cluster center of each of the sets as a third molecule corresponding to the set, each of the third molecules being a representative molecule in a set to which the respective third molecule belongs.

7. The method according to claim 6, wherein the clustering further comprises:
using a Tanimoto coefficient to determine similarity between pairs of the second molecules and clustering a pair of the second molecules into a same one of the sets in response to a determination that the Tanimoto coefficient of the pair is less than a threshold.

8. The method according to claim 1, wherein the performing training comprises:
training a second neural network based on the molecular expression information and the corresponding cost value information of each third molecule in the second cost dictionary;
updating the first cost dictionary based on the second cost dictionary to obtain an updated first cost dictionary;
training a first neural network based on the molecular expression information and the corresponding cost value information in the updated first cost dictionary; and
combining the trained second neural network and the trained first neural network to obtain the target neural network.

9. The method according to claim 8, wherein the training the second neural network comprises:
inputting the molecular expression information of each third molecule in the second cost dictionary into the second neural network to obtain predicted cost value information corresponding to the respective third molecule;
determining a model loss of the second neural network based on the predicted cost value information corresponding to the respective third molecule and the cost value information corresponding to the respective third molecule in the second cost dictionary; and
adjusting a network parameter in the second neural network according to the model loss of the second neural network.

10. A method for determining a molecular retrosynthetic route, the method comprising:
receiving molecular expression information of a target molecule, the molecular expression information representing a three-dimensional chemical structure of the target molecule;
inputting the molecular expression information of the target molecule into a neural network for determining a molecular retrosynthetic route;
determining a disassembly path of the target molecule based on the neural network, the determined disassembly path being a disassembly path with a minimum disassembly cost among at least one possible disassembly path of the target molecule; and
obtaining molecular retrosynthetic route information of the target molecule based on the determined disassembly path.

11. The method according to claim 10, wherein the neural network comprises a first neural network and a second neural network, and the determining the disassembly path comprises:
receiving cost value information of an upper-layer retrosynthetic route of the target molecule as output from the first neural network;
determining a first disassembly path of the target molecule based on the cost value information of the upper-layer retrosynthetic route of the target molecule;
determining molecular expression information of a molecule obtained by disassembling the target molecule based on the first disassembly path; inputting the molecular expression information of the molecule into the second neural network;
receiving cost value information of a lower-layer retrosynthetic route of the target molecule as output from the second neural network;
determining a second disassembly path of the target molecule based on the cost value information of the lower-layer retrosynthetic route of the target molecule; and
determining the disassembly path of the target molecule based on the first disassembly path and the second disassembly path.

12. A training apparatus for a neural network configured to determine a molecular retrosynthetic route, the apparatus comprising:
processing circuitry configured to determine first disassembly paths of a plurality of first molecules such that a first disassembly path is determined for each of the plurality of first molecules based on molecular expression information of the respective one of the plurality of first molecules;
obtain a first cost dictionary based on the first disassembly paths of the first molecules, the first cost dictionary comprising the molecular expression information of each of the first molecules and cost value information corresponding to each of the first molecules, wherein the cost value information of each first molecule represents a cost required to disassemble the respective first molecule according to the first disassembly path of the respective first molecule;
determine molecular expression information of second molecules based on the first disassembly paths of the first molecules, each of the second molecules being a molecule that is obtained by disassembling a corresponding first molecule based on the first disassembly path of the corresponding first molecule, wherein each of the second molecules is capable of being further disassembled;
determine a plurality of third molecules from the second molecules, each of the third molecules representing a class of the second molecules;
obtain a second cost dictionary based on second disassembly paths of the third molecules, the second cost dictionary comprising molecular expression information of each of the third molecules and cost value information corresponding to each of the third molecules, wherein the cost value information of each third molecule represents a cost required to disassemble the respective third molecule according to the second disassembly path of the respective third molecule; and
perform training based on the first cost dictionary and the second cost dictionary to obtain a target neural network, the target neural network being configured to output cost value information corresponding to a target molecule according to input molecular expression information of the target molecule, the cost value information corresponding to the target molecule being used for synthesizing a retrosynthetic route for the target molecule.

13. The apparatus according to claim 12, wherein the processing circuitry is further configured to:
obtain an initial cost value function of each of the first molecules based on the molecular expression information and cost value reference information of the respective first molecule;
in response to a determination that a disassembly level of each of the first molecules is complete, update the initial cost value function of the respective first molecule to obtain a cost value function of each of the respective first molecule based on a disassembly cost value corresponding to the disassembly level of the respective first molecule, the cost value function being used for determining a disassembly path with a minimum disassembly cost value for the respective first molecule; and
in response to a determination that a disassembly task of one of the first molecules satisfies a target disassembly condition, determine a first disassembly path of the one of the first molecules based on the cost value function of the one of the first molecules.

14. The apparatus according to claim 13, wherein the disassembly condition is satisfied when there is no disassembly method for a molecule obtained by the disassembly task of the one of the first molecules, or when a path depth of the disassembly task obtained for the one of the first molecules is equal to a preset depth.

15. The apparatus according to claim 13, wherein the processing circuitry is further configured to:
divide a disassembly task of each of the first molecules into a plurality of first subtasks based on the molecular expression information of the respective first molecule, the disassembly task dividing the respective first molecule according to the disassembly path;
allocate the first subtasks to a plurality of computing nodes, so that the computing nodes calculate and return the initial cost value functions of the respective first molecule, the initial cost value function being calculated by the computing nodes based on molecular cost value reference information representing synthetic accessibility of the respective first molecule; and
receive the initial cost value functions returned by the computing nodes for each of the first molecules.

16. The apparatus according to claim 12, wherein the processing circuitry is further configured to:
determine the cost value information corresponding to each of the first molecules based on the first disassembly path of each of the first molecules; and
obtain the first cost dictionary according to the molecular expression information of each of the first molecules and the cost value information corresponding to each of the first molecules.

17. The apparatus according to claim 12, wherein the processing circuitry is further configured to:
cluster the second molecules to obtain a plurality of sets, each of the sets comprising one or more second molecules with a related molecular structure; and
obtain the plurality of third molecules by respectively determining a cluster center of each of the sets as a third molecule corresponding to the set, each of the third molecules being a representative molecule in a set to which the third molecule belongs.

18. The apparatus according to claim 17, wherein the processing circuitry is further configured to:
cluster the second molecules using a Tanimoto coefficient to determine similarity between pairs of the second molecules and clustering a pair of the second molecules into a same one of the sets in response to a determination that the Tanimoto coefficient of the pair is less than a threshold.

19. The apparatus according to claim 12, wherein the processing circuitry is further configured to:
train a second neural network based on the molecular expression information and the corresponding cost value information of each third molecule in the second cost dictionary;
update the first cost dictionary based on the second cost dictionary to obtain an updated first cost dictionary;
train a first neural network based on the molecular expression information and the corresponding cost value information in the updated first cost dictionary; and
combine the trained second neural network and the trained first neural network to obtain the target neural network.

20. The apparatus according to claim 19, wherein the processing circuitry is further configured to:
input the molecular expression information of each third molecule in the second cost dictionary into the second neural network to obtain predicted cost value information corresponding to the respective third molecule;
determine a model loss of the second neural network based on the predicted cost value information corresponding to the respective third molecule and the cost value information corresponding to the respective third molecule in the second cost dictionary; and
adjust a network parameter in the second neural network according to the model loss of the second neural network.
